(19) United States
(12) Patent Application Publication
(10) Pub. No.: US 2009/0202492 A1
(43) Pub. Date: Aug. 13, 2009

(54) ADENOVIRUS VACCINE UTILIZING IKK AS ADJUVANT

(75) Inventors: AMER AZIZ BEG, Tampa, FL (US); Emily Louise Hopewell, Tampa, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: University of South Florida

(21) Appl. No.: 12/359,935

(22) Filed: Jan. 26, 2009

Related U.S. Application Data
(60) Provisional application No. 61/023,476, filed on Jan. 25, 2008.

(51) Int. Cl. A61K 48/00; C12N 15/861; C12N 15/64; C12N 7/01; C12N 5/10; A61P 31/00

(52) U.S. Cl. 424/93.2; 435/320.1; 435/91.4; 435/235.1; 435/325

(57) ABSTRACT

The invention provides an adenovirus that expresses a constitutively activated form of human NF-κB activating kinase (IKK). Co-administration of the adenovirus expressing the constitutively activated form of IKK (Ad-IKKβ) and an antigen confers an enhanced immune response against the antigen relative to co-administration of the antigen and adenovirus that do not express IKK. Optionally, the adenovirus comprises one or more heterologous nucleic acid sequences that may encode, for example, a biologically active product such as the antigen of an infectious agent (e.g., bacterial, fungal, etc.) or a tumor antigen. Accordingly, the adenovirus is useful for inducing desired immune responses such as those against infectious agents and cancer cells.

### CROSS-REFERENCE TO RELATED APPLICATION

The present application claims the benefit of U.S. Provisional Application Ser. No. 61/023,476, filed Jan. 25, 2008, which is hereby incorporated by reference herein in its entirety, including any figures, tables, nucleic acid sequences, amino acid sequences, and drawings.

### GOVERNMENT SUPPORT

The subject matter of this application has been supported by a research grant from the National Institutes of Health under grant number AI059715. Accordingly, the government has certain rights in this invention.

### BACKGROUND OF THE INVENTION

There is a tremendous need for versatile vaccines that can generate protective immunity against debilitating infectious agents such as HIV, influenza, and hepatitis viruses. Such vaccines may also be used for inducing anti-tumor responses. Adenoviruses can be used as vectors to deliver and express genes in infected cells. In this regard, adenovirus-based vaccines are considered promising candidates.

One way of inducing an immune response in a mammal is by administering an infectious carrier that harbors the antigenic determinant in its genome. One such carrier is a recombinant adenovirus, which has been replication-defective by removal of regions within the genome that are normally essential for replication, such as the E1 region. Examples of recombinant adenoviruses that comprise genes encoding antigens are known in the art (PCT International Patent Publication WO 96/39178), for instance, HIV-derived antigenic components have been demonstrated to yield an immune response if delivered by recombinant adenoviruses (WO 01/02607 and WO 02/22080).

Two IκB kinases (IKK1 or IKK2, also known as IKKα or IKKβ, respectively), which phosphorylate IκB and thereby initiate their degradation, have been cloned and characterized by a number of laboratories (Khuynh Q. K. et al., The Journal of Biological Chemistry, 2000, 275(34):25883-25891; Regnier, C. et al., Cell, 1997, 90:373-383; DiDoonato J. A. et al., Nature, 1997, 388:548-554; Mercurio F. et al., Science, 1997 278:860-866; Zandi E. et al., Cell, 1997, 91:243-252; Woronicz, J. D. et al., Science, 1997, 278:866-869). Using airway delivery of replication-deficient adenovirus that express a constitutively activated form of IκB kinase 1 or IκB kinase 2, it has been demonstrated that constitutive expression of IKK1 or IKK2 in lung epithelium causes sufficient activation of NF-κB to direct cytokine expression and generate neutrophilic lung inflammation (Sadikot R. T. et al., The Journal of Immunology, 2003, 170:1091-1098).

### BRIEF SUMMARY OF THE INVENTION

NF-κB plays a critically important role in expression of multiple genes that stimulate immunity. NF-κB is a transcription factor involved in cellular response to a challenge from stress, radiation, bacterial or viral insult, and cytokines. While inactive, NF-κB is bound to IκB proteins, which sequester NF-κB to the cytoplasm. During a cellular challenge, IκB is phosphorylated by IκB kinase (IKK) leading to ubiquitination and proteolysis of IκB, freeing NF-κB. NF-κB enters the nucleus, where it activates NF-κB-mediated genes.

This invention relates to immune response regulators. The present invention provides an adenoviral vector that expresses a constitutively activated form of the NF-κB activating kinase, (IKK), also referred to herein as Ad-IKK. In some embodiments, the constitutively activated form of IKK expressed by the adenovirus is IKKα (Ad-IKKα). In some embodiments, the constitutively activated form of IKK expressed by the adenovirus is IKKβ (Ad-IKKβ). The adenovirus can be replication-competent or replication-deficient. Preferably, the adenovirus is replication-deficient.

Ad-IKK can be used to express products such as target antigens (Ag) against which an immune response is sought. The present inventors have found that co-administration of the adenovirus expressing the constitutively activated form of IKK (Ad-IKKβ) and an antigen confers an enhanced immune response against the antigen relative to co-administration of the antigen and adenovirus that do not express IKK. Therefore, it is expected that after administration of Ad-IKK-Ag virus (i.e., a virus that encodes the antigen), a stronger immune response against the target Ag will be elicited as compared to Ag expressed by Ad without IKK (Ad-Ag). Advantageously, virtually any target Ag can be used with this vector system. These can include Ags expressed in tumors or those encoded by infectious agents such as virus and bacteria. Therefore, there is tremendous potential for use of Ad-IKK in many situations. Importantly, Ad-IKK is useful even without antigens. For example, direct administration (e.g., local injection) of Ad-IKK to tumors can create a more immunogenic environment and thus enhance the ability of T cells or other cell types to kill the tumor cells.

In one aspect, the invention provides an adenoviral vector comprising a recombinant adenovirus that expresses a constitutively activated form of human NF-κ B activating kinase (IKKα or IKKβ), and comprises one or more heterologous nucleic acid sequences that encode a biologically active product such as a biologically active polypeptide or interfering RNA molecule (e.g., siRNA). In some embodiments, the heterologous nucleic acid sequence encodes a biologically active polypeptide, such as the antigen of an infectious agent (e.g., bacterial, fungal, etc.) or a tumor antigen.

Another aspect of the invention is an immunogenic composition comprising an adenovirus that expresses a constitutively activated form of human IKK (IKKα or IKKβ), and an antigen against which an immune response is desired, and/or an adjuvant or immunogen. In some embodiments, the antigen is a protein. The adjuvant/immunogen can be a live attenuated vaccine, killed vaccine, toxoid vaccine, component vaccine, recombinant virus vaccine, anti-idiotype antibody, DNA vaccine, or immuno-stimulatory microbial product(s), for example. In embodiments in which the composition includes host cells comprising the adenovirus, the host cells may be, for example, immuno-stimulatory cells, such as dendritic cells, that have been infected with the adenovirus. In some embodiments of the immunogenic composition, the adenovirus comprises a heterologous nucleic acid encoding a biologically active product, such as an antigen, adjuvant, or immunogen, or non-biologically active product. In some embodiments of the immunogenic composition, the adenovirus comprises no heterologous nucleic acids other than the IKK mutation.

Another aspect of the invention is a method for inducing an immune response in mammal such as a human, comprising administering an effective amount of an adenovirus expressing a constitutively activated form of human IKK (IKKα or IKKβ) to the mammal, or administering an effective amount of host cells comprising Ad-IKK. For example, the adenovirus of the invention (Ad-IKK) can be used as a vaccine adjuvant allowing heightened immune response following antigen insult. Thus, the method for inducing an immune response can further comprise administration of an effective amount of an antigen, immunogen, and/or adjuvant before, during, or after administration of the Ad-IKK. The adjuvant/immunogen can be a vaccine, such as a live attenuated vaccine, killed vaccine, toxoid vaccine, component vaccine, recombinant virus vaccine, anti-idiotype antibody, DNA vaccine, or immuno-stimulatory microbial product(s), for example. The antigen can be administered to the subject in separately from the Ad-IKK or within the same composition.

In some embodiments of the method for inducing an immune response, an effective amount of host cells comprising the adenovirus (Ad-IKK) are administered to the subject. For example, immuno-stimulatory cells such as dendritic cells can be infected with Ad-IKK and administered to the mammal, e.g., as a vaccine. Preferably, the cells are the subject's own cells, but may be allogeneic or xenogeneic. In some embodiments, the method further comprises administration of an effective amount of an antigen (such as an immunogen) before, during, or after administration of the cells. The antigen can be administered to the subject separately from the cells or within the same composition.

In some embodiments of the method for inducing an immune response, the adenovirus (Ad-IKK) that is administered to the subject directly or administered to the subject via host cells further comprises a heterologous nucleic acid encoding a biologically active product or non-biologically active product. In some embodiments of the method for inducing an immune response, the Ad-IKK comprises no heterologous nucleic acids other than the IKKβ mutation and operably linked elements (e.g., CMV promoter).

Another aspect of the invention is a method for producing an adenovirus expressing a constitutively activated form of human IKK (IKKα or IKKβ), comprising inducing mutations in an adenovirus such that the adenovirus expresses a constitutively activated form of human NF-κB activating kinase (IKK); and introducing a heterologous nucleic acid sequence into the adenovirus, wherein the heterologous nucleic acid sequence encodes a biologically active product.

A method of producing an immunogenic composition, comprising combining an adenovirus comprising a mutation that expresses a constitutively activated form of human NF-κB activating kinase (IKKα or IKKβ), and an antigen against which an immune response is desired, and/or an immunogen or adjuvant. The immunogen can be a vaccine, such as a live attenuated vaccine, killed vaccine, toxoid vaccine, component vaccine, recombinant virus vaccine, anti-idiotype antibody, DNA vaccine, or immuno-stimulatory microbial product(s), for example.

Another aspect of the invention is a viral particle comprising an adenovirus expressing a constitutively activated form of human IKK (IKKα or IKKβ), and expresses a heterologous nucleic acid sequence encoding a biologically active molecule.

Another aspect of the invention is an isolated host cell comprising an adenovirus expressing a constitutively activated form of human IKK (IKKα or IKKβ), and expresses a heterologous nucleic acid sequence encoding a biologically active molecule, such as an antigen. In some embodiments, the host cell is a mammalian cell. In some embodiments, the host cell is a human cell. In some embodiments, the host cell is the cell of a cell line (e.g., 293 cells, HEK cells). Another aspect of the invention is an isolated immuno-stimulatory cell such as a dendritic cell comprising an adenovirus expressing a constitutively activated form of human IKK (Ad-IKKα or Ad-IKKβ). The dendritic cell may be infected with Ad-IKK and administered to a subject, e.g., as a vaccine. Optionally, the Ad-IKK further comprises a heterologous nucleic acid sequence encoding a biologically active product, such as an antigen, or a non-biologically active product such as a reporter molecule.

### BRIEF DESCRIPTION OF THE DRAWINGS

FIGS. 1A and 1B are blots showing expression of IKKβ protein after infection with adenovirus constructs. FIG. 1A shows expression of IKKβ in 293T cells after Ad or Ad-IKK infection at a multiplicity of infection (MOI) of 20 for 48 hours. FIG. 1B shows expression in murine bone marrow-derived DCs at an MOI of 100 or 260, as indicated, for 48 hours.

FIG. 2 is a blot depicting activation of NF-κB in 293T cells after Ad or Ad-IKK infection shown by electrophoretic mobility shift assay. 293T cells were infected at varying MOI, as indicated, for 48 hours.

FIGS. 3A and 3B are a series of blots showing mRNA expression of immuno-stimulatory cytokines in DCs after Ad, Ad-IKK, or Ad-Gal4 infection as shown by RPA. In FIG. 3A, murine bone marrow-derived DCs were infected with Ad constructs at an MOI of 250 for 28 hours. Ad-Gal4 served as a control vector containing the Gal4 reporter gene instead of the constitutively active IKK gene, In FIG. 3B, human monocyte-derived DCs were infected with Ad constructs at an MOI of 500 for 48 hours.

FIGS. 4A and 4B are graphs depicting protein levels in supernatant from murine BMDC infected with Ad or Ad-IKK for 48 hours at an MOT of 250. FIG. 4A shows levels of IL-12p70 in pg/ml as determined by ELISA. FIG. 4B shows levels of IL-6 in ng/ml as determined by ELISA.

FIGS. 5A and 5B are graphs showing humoral and cell-mediated immune responses of mice to co-administration of Ad-IKK and antigen. FIG. 5A shows sera of mice (n=2 per condition) injected s.c. with 5e8 PFU Adenovirus+100 μg ova showed a significant increase in anti-ova IgG for Ad-IKK compared to Ad-GFP injected mice after 21 days (serum dilution: 5000×). FIG. 5B shows CD8 T cell response of mice (n=2 per condition) injected as in (b) showed a ˜4-fold increase in IFNγ-producing cells in Ad-IKK mice compared to Ad-GFP injected mice after 8 days as shown by ELISPOT.

### DETAILED DESCRIPTION OF THE INVENTION

In one aspect, the invention provides an adenoviral vector comprising a recombinant adenovirus that expresses a constitutively activated form of human NF-κB activating kinase (IKKα or IKKβ). Preferably, the IKKβ is rendered constitutively activated by mutations in serines at amino acid residues 177 and 181 in the adenovirus (Mercurio, F. et al., Science, 1997, 278:860). Preferably, the IKKα is rendered constitutively activated by mutations in serines at amino acid residues 176 and 180 in the adenovirus. In some embodiments, IKK is rendered constitutively activated by substitution of the indicated serines with glutamic acid or aspartic acid. Thus, for example, IKKβ can be rendered constitutively activated by the substitutions S177E and S181E, or S177D and S181D, or S177E and S181D, or S177D and S181E. Likewise, IKKα can be rendered constitutively activated by the substitutions S176E and 180E, or S176D and S180D, or S176E and S180D, or S176D and S176E. The adenovirus can be replication-competent or replication-deficient. Preferably, the adenovirus is replication-deficient.

Optionally, the adenovirus comprises one or more heterologous nucleic acid sequences that may encode, for example, a polypeptide or interfering RNA molecule (e.g. siRNA). The adenovirus can be provided with an appropriate expression control sequence operably linked to the heterologous nucleic acid sequence. In some embodiments, the heterologous nucleic acid sequence encodes a polypeptide, such as the antigen of an infectious agent (e.g., bacterial, fungal, etc.) or a tumor antigen. In some embodiments, the adenovirus comprises no heterologous nucleic acid sequences other than the IKK mutation.

Another aspect of the invention is an immunogenic composition comprising: (a) an adenovirus that expresses a constitutively activated form of human IKK (IKKα or IKKβ), and (b) an antigen, immunogen, and/or adjuvant. In some embodiments, the antigen is a protein antigen. The immunogen can be a vaccine, such as a live attenuated vaccine, killed vaccine, toxoid vaccine, protein subunit or component vaccine, recombinant virus vaccine, anti-idiotype antibody, DNA vaccine, or immuno-stimulatory microbial product, for example. Examples of killed (inactivated) vaccines include inactivated influenza vaccine (IFV), cholera, bubonic plague, inactivated polio vaccine (IPV), and hepatitis A. Examples of live attenuated vaccines include, but are not limited to, yellow fever, measles, rubella, mumps, oral polio vaccine, Varicella (chicken pox), and tuberculosis (BCG strain). Examples of toxoid vaccines include, but are not limited to, tetanus, diphtheria, and Crotalis atrox toxoid (to vaccinate dogs against rattle snake bites). Examples of subunit vaccines include, but are not limited to, the surface proteins of Hepatitis B virus, the virus-like particle (VLP) vaccine against human papillomavirus (HPV) composed of the viral major capsid protein.

Another aspect of the invention is a method for inducing an immune response in a mammal such as a human, comprising administering an effective amount of an adenovirus expressing a constitutively activated form of human IKK (IKKα or IKKβ) to the mammal, wherein an immune response is induced in the mammal. The induced immune response can comprise a cell-mediated immune response, humoral immune response, or both. For example, the adenovirus of the invention can be used as a vaccine adjuvant allowing heightened immune response following antigen insult. Thus, the adenovirus of the invention can be administered to a mammal before, during, or after administration of an antigen, immunogen, and/or adjuvant. The immunogen can be a vaccine, such as a live attenuated vaccine, killed vaccine, toxoid vaccine, component vaccine, recombinant virus vaccine, anti-idiotype antibody, DNA vaccine, or immuno-stimulatory microbial product, for example. The antigen, immunogen, and/or adjuvant can be administered to the subject separately from the adenovirus or within the same composition.

The method for inducing an immune response can be used as treatment for a disorder, such as a hyperproliferative disorder or an infection, or the method can be used to raise antibodies in the mammal, for example. Methods for isolating raised antibodies from mammals such as mouse, rabbit, horse, and goat, and using such antibodies for research, or within the clinical setting, are known in the art. Subjects in need of treatment using the methods of the invention (methods for inducing an immune response) can be identified using standard techniques known to those in the medical or veterinary professions.

The methods of inducing an immune response may comprise administration of about 10³ to about 10¹⁵ adenoviral particles to the subject. More preferably about 10⁵ to about 10¹² adenoviral particles, and most preferably about 10⁸ to about 10¹² adenoviral particles are administered to the subject.

The target cells of the adenovirus may be located, for example, in a human subject's nervous system, circulatory system, digestive system, respiratory system, reproductive system, endocrine system, skin, muscles, or connective tissue. In veterinary applications, similar target cells would be applicable. The target cells of the adenovirus include any mammalian host cell. In particular, target cells can be dendritic cells, tumor cells, virus-infected cells, bacteria-infected cells, or cells causing genetically based disease. The target cells may have surface markers which are inherently present or which are present due to a disease condition. These surface markers may include specific receptors, or selective antigens, such as tumor-associated antigens. The type and number of surface markers of a cell provide a unique profile to that cell, distinguishing a given cell from other cells present in the host.

In some embodiments, the methods of inducing an immune response may further comprise administering to the subject a second therapy, such as chemotherapy, immunotherapy (e.g., antibodies), surgery, radiotherapy, biological therapy, cryotherapy, hyperthermia, ultrasound, immunosuppressive agents, or a gene therapy with a therapeutic polynucleotide, before, during, or after administration of the adenovirus or cells infected with the adenovirus. The second therapy may be administered to the subject before, during, after or concurrently with administration of the adenovirus of the invention. Chemotherapy can comprise, for example, an alkylating agent, mitotic inhibitor, antibiotic, or antimetabolite. In some embodiments, the chemotherapy comprises CPT-11, temozolomide, taxanes or a platin compound. Radiotherapy may comprise, for example, X-ray irradiation, UV-irradiation, gamma-irradiation, or microwaves. Accordingly, the immunogenic compositions of the invention may also include active agents for administering such therapies.

The hyperproliferative disorder treated by administration of the adenovirus of the invention may be a precancerous condition, such as cellular hyperplasia, adenoma, metaplasia, or dysplasia, for example, or abnormal skin growth such as warts caused by viral infection such as human papillomavirus infection (e.g., common wart, flat wart, filiform or digitate wart, plantar wart, mosaic wart, genital wart). In other embodiments, the hyperproliferative disorder is cancer, such as a carcinoma, a sarcoma, a metastatic cancer, a lymphatic metastases, a blood cell malignancy, a multiple myeloma, an acute leukemia, a chronic leukemia, a lymphoma, a head and neck cancer, a mouth cancer, a larynx cancer, a thyroid cancer, a respiratory tract cancer, a lung cancer, a small cell carcinoma, a non-small cell cancer, a breast cancer, ductal carcinoma, gastrointestinal cancer, esophageal cancer, stomach cancer, colon cancer, colorectal cancer, pancreatic cancer, liver cancer, genitourinary cancer, urologic cancer, bladder cancer, prostate cancer, ovarian carcinoma, uterine cancer, endometrial cancer, kidney cancer, renal cell carcinoma, brain cancer, neuroblastoma, astrocytic brain tumors, gliomas, metastatic tumor cell invasion in the central nervous system, bone cancers, osteomas, skin cancer, malignant melanoma, squamous cell carcinoma, basal cell carcinoma, hemangiopericytoma or Kaposi's sarcoma. The cancer may be a recurrent cancer, a refractory cancer, a primary cancer, or a metastasis.

The adenovirus can include one or more heterologous nucleic acid sequences encoding one or more products, such as polypeptides. The polypeptide product thus encoded can be a protein, a peptide, and the like. In some embodiments, the product encoded by the heterologous nucleic acid sequence is biologically active.

In some embodiments, the product encoded by the heterologous nucleic acid sequence is not biologically active. For example, in some embodiments, the non-biologically active product encoded by the exogenous nucleic acid sequence is a reporter molecule, such as green fluorescent protein (GFP) or other reporter molecule (e.g., luciferase, β-galactosidase) that is used to identify infected cells or transgene expression.

In some embodiments, the product encoded by the heterologous nucleic acid sequence is biologically active and has a therapeutic or prophylactic effect. This product can be homologous with respect to the target cell (that is to say a product that is normally expressed in the target cell when the latter is not suffering from any pathology). In this case, the expression of a product makes it possible, for example, to remedy an insufficient expression in the cell or the expression of a gene product (e.g., protein) which is inactive or weakly active on account of a genetic abnormality, or alternatively to over-express the protein. Optionally, the biologically active product may encode a variant of a cell protein, having enhanced stability, modified activity, and the like. The biologically active product may also be heterologous with respect to the target cell. In this case, an expressed product may, for example, supplement or supply an activity which is deficient in the cell, enabling it to combat a pathology, or stimulate or enhance an immune response (e.g., against an infectious agent such as a virus, bacteria, fungus, etc.). The therapeutic nucleic acid sequence may also code for a polypeptide or other product secreted by the cell into the body.

The heterologous nucleic acid sequence can encode fusion polypeptides or multimeric peptides (see, for example, Fooks, A. R. et al., Journal of General Virology, 1998, 1027-1031).

Examples of biologically active molecules that may be encoded by the heterologous nucleic acid sequence(s) included within the adenovirus include, but are not limited to, enzymes; blood derivatives; hormones; lymphokines, namely interleukins, interferons, tumor necrosis factor, and the like; growth factors; neurotransmitters or their precursors or synthetic enzymes; trophic factors, namely BDNF, CNTF, NGF, IGF, GMF, alpha-FGF, beta-FGF, NT3, NT5, HARP/pleiotrophin, and the like; apolipoproteins, namely ApoAI, ApoAIV, ApoE, and the like; dystrophin or a minidystrophin; the CFTR protein associated with cystic fibrosis; intrabodies. Additional examples of nucleic acid sequences include tumor-suppressing genes, namely p53, Rb, Rap1A, DCC, k-rev, and the like; genes coding for factors involved in coagulation, namely factors VII, VIII, IX; genes participating in DNA repair; suicide genes (genes whose products cause the death of a cell; e.g., thymidine kinase (HS-TK), cytosine deaminase), and the like; pro-apoptic genes; prodrug converting genes (genes coding for enzymes who convert prodrugs to drugs); and anti-angiogenic genes, or alternatively, genes such as VEGF that promote angiogenesis.

The biologically active product encoded by the heterologous nucleic acid sequence can be an antigen useful for stimulating or enhancing an immune response (e.g., humoral and/or cell-mediated immune response) against an infectious agent such as a bacteria, virus, etc. For example, the nucleic acid sequence(s) can encode one or more antigens of microorganisms (immuno-stimulatory microbial products), including pathogens, such as HIV, influenza, and hepatitis virus.

Various methodologies and expression control sequences (e.g., promoters) are available for delivering and expressing a polynucleotide in vivo for the purpose of treating disorders such as cancer (Robson, T. Hirst, D. G., J. Biomed. and Biotechnol., 2003, 2003(2): 110-137). Among the various expression targeting techniques available, transcriptional targeting using tissue-specific and event-specific transcriptional control elements may be used. For example, several tissue-specific promoters, tumor environment-specific promoters, and exogenously controlled inducible promoters, are known in the art, which may be used with the adenovirus of the invention.

The adenovirus can include a heterologous nucleic acid sequence encoding an interfering RNA molecule, such as siRNA or shRNA, which targets a gene for knockdown or silencing (partial or complete inhibition of gene expression). The gene targeted for knockdown may be, for example, a gene endogenous to the mammal to which the adenovirus is administered, such as the gene of a cancer cell, or the gene of an infectious agent, such as the gene of a virus or bacteria (e.g., a gene necessary for the infectious agent's survival, replication, spread, etc.). Methods for the production and delivery of adenovirus expressing small interfering RNAs or short hairpin RNAs are known in the art (see, for example, Arts, (G-J. et al., Genome Res., 2003, 13:2325-2332; Shen C. and Reske S. N., Methods Mol. Biol., 2004, 252:523-532; Kuninger D. et al., Human Gene Therapy, 2004, 15(12):1287-1292; Huang D. et al., Journal of U.S.-China Medical Science, 2005, 2(4, Serial No. 5):62-69; Bain J. R. et al., Diabetes, 2004, 53:2190-2194; Zhao L-J. et al., Gene, 2003, 316:137-141; Li H. et al., Gastroenterology, 2005, 128(7):2029-2041; Carette J. E., Cancer Research, 2004, 64:2663-2667; Huang B. and S. Kochanek, Human Gene Therapy, 2005, 16(5):618-626).

For example, the RNAi molecule can target collagen, cyclin dependent kinase, cyclin D1, cyclin E, WAF 1, cdk4 inhibitor, MTS 1, cystic fibrosis transmembrane conductance regulator, IL-1, IL-2, IL-3, IL-4, IL-5, IL-6, IL-7, IL-8, IL-9, IL-10, IL-11, IL-12, IL-13, IL-14, IL-15, IL-16, IL-17, erythropoietin, G-CSF, GM-CSF, M-CSF, SCF, thrombopoietin, BNDF, BMP, GGRP, EGF, FGF, GDNF, GGF, HGF, IGF-1, IGF-2, KGF, myotrophin, NGF, OSM, PDGF, somatotrophin, TGF-beta, TGF-alpha, VEGF, interferon, TNF-alpha, TNF-beta, cathepsin K, cytochrome p-450, farnesyl transferase, glutathione-s transferase, heparanase, HMG CoA synthetase, n-acetyltransferase, phenylalanine hydroxylase, phosphodiesterase, ras carboxyl-terminal protease, telomerase, TNF converting enzyme, E-cadherin, N-cadherin, selectin, CD40, 5-alpha reductase, atrial natriuretic factor, calcitonin, corticotrophin releasing factor, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, thyroid stimulating hormone, immunoglobulin, CTLA4, hemagglutinin, major histocompatibility factor (MHC), VLA-4, kallilkrein-kininogen-kinin system, CD4, sis, hst, ras, abl, mos, myc, fos, jun, H-ras, ki-ras, c-fms, bcl-2, L-myc, c-myc, gip, gsp, HER-2, bombesin receptor, estrogen receptor, GABA receptor, EGFR, PDGFR, FGFR, NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors, tissue plasminogen activator; transmembrane receptors, calcium pump, proton pump, Na/Ca exchanger, MRP 1, MRP2, P170, LRP, cMOAT, transferrin, APC, brca1, brca2, DCC, MCC, MTS1, NF1, NF2, nm23, p53, or Rb within the host cell in vitro or in vivo. In some embodiments, the interfering RNA targets a gene that encodes an oncogene, a transcription factor, a receptor, an enzyme, a structural protein, an cytokine, a receptor, a cytokine receptor, a lectin, a selectin, an immunoglobulin, a kinase or a phosphatase. In some embodiments, the interfering RNA targets one or more oncogene or tumor suppressor gene selected from bcl-2, bcr-abl, bek, BPV, c-abl, c-fes, c-fms, c-fos, c-H-ras, c-kit, c-myb, c-myc, c-mos, c-sea, cerbB, DCC, erbA, erbB-2, ets, fig, FSFV gp55, Ha-ras, HIV tat, HTLV-1 tat, JCV early, jun, L-myc, lck, LPV early, met, N-myc, NF-1, N-ras, neu, p53, Py mTag, pim-1, ras, RB, rel, retinoblastoma-1, SV-40 Tag, TGF-alpha, TGF-beta, trk, trkB, v-abl, v-H-ras, v-jun, or WT-1.

In some embodiments, the interfering RNA targets a gene that encodes an oncogene, a transcription factor, a receptor, an enzyme, a structural protein, an amyloid protein, amyloid precursor protein, angiostatin, endostatin, METH-1, METH-2, Factor IX, Factor VIII, collagen, cyclin dependent kinase, cyclin D1, cyclin E, WAF 1, cdk4 inhibitor, MTS1, cystic fibrosis transmembrane conductance regulator, IL-1, IL-2, IL-3, IL-4, IL-5, IL-6, IL-7, IL-8, IL-9, IL-10, IL-11, IL-12, IL-13, IL-14, IL-15, IL-16, IL-17, erythropoietin, G-CSF, GM-CSF, M-CSF, SCF, thrombopoietin, BNDF, BMP, GGRP, EGF, FGF, GDNF, GGF, HGF, IGF-1, IGF-2, KGF, myotrophin, NGF, OSM, PDGF, somatotrophin, TGF-beta, TGF-alpha, VEGF, interferon, TNF-alpha, TNF-beta, cathepsin K, cytochrome p-450, farnesyl transferase, glutathione-s transferase, heparanase, HMG CoA synthetase, n-acetyltransferase, phenylalanine hydroxylase, phosphodiesterase, ras carboxyl-terminal protease, telomerase, TNF converting enzyme, E-cadherin, N-cadherin, selectin, CD40, 5-alpha reductase, atrial natriuretic factor, calcitonin, corticotrophin releasing factor, glucagon, gonadotropin, gonadotropin releasing hormone, growth hormone, growth hormone releasing factor, somatotropin, insulin, leptin, luteinizing hormone, luteinizing hormone releasing hormone, parathyroid hormone, thyroid hormone, thyroid stimulating hormone, immunoglobulin, CTLA4, hemagglutinin, major histocompatibility factor (MHC), VLA-4, kallilkrein-kininogen-kinin system, CD4, sis, hst, ras, abl, mos, myc, fos, jun, H-ras, ki-ras, c-fins, bcl-2, L-myc, c-myc, gip, gsp, HER-2, bombesin receptor, estrogen receptor, GABA receptor, EGFR, PDGFR, FGFR, NGFR, GTP-binding regulatory proteins, interleukin receptors, ion channel receptors, leukotriene receptor antagonists, lipoprotein receptors, opioid pain receptors, substance P receptors, retinoic acid and retinoid receptors, steroid receptors, T-cell receptors, thyroid hormone receptors, TNF receptors, tissue plasminogen activator; transmembrane receptors, calcium pump, proton pump, Na/Ca exchanger, MRP 1, MRP2, P170, LRP, cMOAT, transferrin, APC, brca1, brca2, DCC, MCC, MTS1, NF1, NF2, or nm23. In some embodiments, the interfering RNA targets a Ras protein, p53, pRb, EF2-1, bcl-2, bcr-abl, bek, BPV, c-abl, c-fes, c-fms, c-fos, c-H-ras, c-kit, c-myb, c-myc, c-mos, c-sea, cerbB, DCC, erbA, erbB-2, ets, fig, FSFV gp55, Ha-ras, HIV tat, HTLV-1 tat, JCV early, jun, L-myc, lck, LPV early, met, N-myc, NF-1, N-ras, neu, p53, Py mTag, pim-1, ras, RB, rel, retinoblastoma-1, SV-40 Tag, TGF-alpha, TGF-beta, trk, trkB, v-abl, v-H-ras, v-jun, or WT-1.

The adenovirus of the invention may be administered to a mammalian subject locally at the desired anatomical site (e.g., the site of a tumor) or systemically. For example, the adenovirus can be administered orally, intratracheally, parenterally (e.g., intravascularly such as intravenously), intramuscularly, sublingually, buccally, rectally, intranasally, intrabronchially, intrapulmonarily, intraperitonealy, topically, transdermally and subcutaneously, for example. In some embodiments, the adenovirus is administered to the subject by a route other than intratracheally. Host cells may be administered to the subject by any effective route, such as orally, intracheally, parenterally (e.g., intravascularly such as intravenously), intramuscularly, intracranially, intracerebrally, intradermally, intraocularly, nasally, topically, or by open surgical procedure. In some embodiments, the host cells are administered to the subject by a route other than intratracheally.

The adenovirus may be administered to a subject once or as a regimen of multiple doses. The amount of adenovirus administered in a single dose may be dependent on the subject being treated, the subject's weight, the manner of administration and the judgment of the prescribing physician. Generally, however, administration and dosage and the duration of time for which the adenovirus is administered will approximate that which is necessary to achieve a desired result. Each dose of adenovirus may be administered to a subject in combination with a pharmaceutically acceptable carrier and, optionally, may include other medicinal agents, pharmaceutical agents, carriers, adjuvants, diluents, etc.

The adenovirus may be formulated in a composition using methods known in the art. Formulations are described in a number of sources which are well known and readily available to those skilled in the art. For example, Remington's Pharmaceutical Science (Martin, E. W., 1995, Easton Pa., Mack Publishing Company, 19^{th} ed.) describes formulations which can be used in connection with the subject invention. Formulations suitable for administration include, for example, aqueous sterile injection solutions, which may contain antioxidants, buffers, bacteriostats, and solutes that render the formulation isotonic with the blood of the intended recipient; and aqueous and nonaqueous sterile suspensions which may include suspending agents and thickening agents. The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the compositions of the subject invention can include other agents conventional in the art having regard to the type of formulation in question.

In some embodiments, the compositions of the invention include an adjuvant. The adjuvant selected typically depends on the subject receiving the composition, e.g., the mammal being used to generate antibodies. Different adjuvants produce different responses in different animals. Some adjuvants are inappropriate for certain animals, due to the inflammation, tissue damage, and pain that are caused to the animal. Other factors that influence the choice of an adjuvant include the injection site, the manner of antigen preparation, and amount of antigen injected. One type of adjuvant that may be used and that has been of long-standing service in generating antibodies for the study of bacteria is known as Freund's Complete Adjuvant. This type of adjuvant enhances the response to the antigen of choice via the inclusion mycobacteria into a mixture of oil and water. Sometimes the mycobacteria are left out of the adjuvant. In this case, it is referred to as “incomplete” adjuvant.

Another aspect of the invention is a method for producing an adenovirus expressing a constitutively activated form of human IKK, comprising providing an adenovirus, and genetically modifying the adenovirus such that the adenovirus expresses a constitutively activated form of human IKK, and expresses a heterologous nucleic acid sequence encoding a biologically active molecule, such as an antigen. In some embodiments, the Ad-IKK is obtained and genetically modified with the heterologous nucleic acid sequence. In some embodiments, an adenovirus comprising the heterologous nucleic acid sequence is obtained, and genetically modified to express the constitutively activated form of IKK.

Another aspect of the invention is a viral particle comprising an adenovirus that expresses a constitutively activated form of human IKK, and expresses a heterologous nucleic acid sequence encoding a biologically active product, such as an antigen.

Another aspect of the invention is an isolated host cell comprising an adenovirus expressing a constitutively activated form of human IKK, and expressing a heterologous nucleic acid sequence encoding a biologically active product, such as an antigen. In some embodiments, the host cell is a mammalian cell. In some embodiments, the host cell is a human cell. In some embodiments, the host cell is the cell of a cell line (e.g., 293 cells, HEK cells). Another aspect of the invention is an isolated immuno-stimulatory cell such as a dendritic cell comprising an adenovirus expressing a constitutively activated form of human IKK (Ad-IKKα or Ad-IKKβ). The dendritic cell may be infected with Ad-IKK and administered to a subject, e.g., as a vaccine. Optionally, the Ad-IKK further comprises a heterologous nucleic acid sequence encoding a biologically active or non-biologically active product.

Another aspect of the invention is a method for producing a host cell, comprising administering to a cell an effective amount of adenovirus expressing a constitutively activated form of human IKK, and expressing a heterologous nucleic acid sequence encoding a biologically active product, such as antigen. The Ad-IKK may be administered to the cell in vitro, or administered in vivo and subsequently isolated.

Another aspect of the invention is a method for producing an immuno-stimulatory cell, comprising administering to an immuno-stimulatory cell, such as a dendritic cell, an effective amount of adenovirus expressing a constitutively activated form of human IKK (Ad-IKK). Optionally, the Ad-IKK has also been genetically modified to express a heterologous nucleic acid sequence encoding a biologically active product, such as antigen, or a non-biologically active product, such as a reporter molecule. The Ad-IKK may be administered to the cell in vitro, or administered in vivo and subsequently isolated. Adenovirus (Ad) is a 36 kb double-stranded DNA virus that efficiently transfers DNA in vivo to a variety of different target cell types. The adenoviral vector can be produced in high titers and can efficiently transfer DNA to replicating and non-replicating cells. The adenoviral vector genome can be generated using any species, strain, subtype, mixture of species, strains, or subtypes, or chimeric adenovirus as the source of vector DNA. Adenoviral stocks that can be employed as a source of adenovirus can be amplified from the human adenoviral serotypes 1 through 51, which are currently available from the American Type Culture Collection (ATCC, Manassas, Va.), or from any other serotype of human adenovirus available from any other source. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22-30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. Given that the human adenovirus serotype 5 (Ad5) genome has been completely sequenced, the adenoviral vector of the invention is described herein with respect to the Ad5 serotype; however, other adenovirus serotypes may be used. The complete genome of wild-type Ad5 can be found, for example, at GenBank accession numbers AC_{—}000008 and BK000408 (Chroboczek, J. et al., J. Gen. Virol., 2003, 84 (Pt 11), 2895-2908; Davison, A. J. et al., J. Gen. Virol., 2003, 84 (Pt 11), 2895-2908). In addition to human adenovirus, the adenovirus of the invention can be generated using a non-human primate adenovirus, in which case, the adenovirus is preferably a chimpanzee adenovirus.

Adenoviral vectors are well known in the art and are described in, for example, U.S. Pat. Nos. 5,559,099; 5,712,136; 5,731,190; 5,837,511; 5,846,782; 5,851,806; 5,962,311; 5,965,541; 5,981,225; 5,994,106; 6,020,191; 6,083,716; 6,113,913; and 6,482,616; U.S. Patent Application Publication Nos. 2001/0043922 A1; 2002/0004040 A1; 2002/0031831 A1; and 2002/0110545 A1; International Patent Applications WO 95/34671; WO 97/21826; and WO 00/00628; and Thomas Shenk, “Adenoviridae and their Replication,” and M. S. Horwitz, “Adenoviruses,” Chapters 67 and 68, respectively, in Virology, B. N. Fields et al., eds., 3d ed., Raven Press, Ltd., New York (1996); and Fooks A. R., Live Viral Vectors: Construction of a Replication-Deficient Recombinant Adenovirus, in Methods in Molecular Medicine, Vol. 87, Vaccine Protocols, Second Edition, Edited by A. Robinson, M. J. Hudson, and M. P. Cranage, Humana Press Inc. Totowa N.J., pages 37-50 (2003). Adenoviral vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994).

Techniques for the production and delivery of adenoviral vectors as gene carriers and vaccines are known in the art (see, for example, Bruce, C. B. et al., J. Gen. Virol., 1999, 80:2621-2628; Wildner O. et al., Gene Therapy, 1999, (6)57-62; Stahl-Hennig C. et al., J. Virol., 2007, 81(23):13180-13190; Fooks A. R. et al., J. Gen. Virol., 1998, 79(Part 5):1027-1031; Shiver J. W. et al., Nature, 2002, 415(6869):331-335; Wang J. et al, J Immunol., 2004, 173:6357-6365; Lin S. W. et al., Vaccine, 2007, 25(12):2187-2193; Shanley J. D. and Wu, C. A., Vaccine, 2005, 23(8):996-1003; Shanley J. D. and Wu, C. A., Vaccine, 2003, 21(19-20):2632-2642; Sharpe S. et al., Virology, 2002, 293(2):210-216(7); Caetano B. C. et al., Hum Gene Ther., 2006, 17(4):415-426; Pergolizzi R. G. et al., Hum. Gene Ther., 2005, 16(3):292-298; international patent publication WO 1996/039178 (Ertl H. and Wilson, J. M.); international patent publication WO 2001/002607 (Chen L. et al.); international patent publication WO 2001/021201 (Schneider J. et al.); and U.S. Pat. No. 5,698,202 (Ertl H. et al.).

### I. DEFINITIONS

In order that the present disclosure may be more readily understood, certain terms are first defined. Additional definitions are set forth throughout the detailed description.

“A” or “an,” as used herein in the specification, may mean one or more than one. As used herein in the claim(s), when used in conjunction with the word “comprising,” the words “a” or “an” may mean one or more than one. Thus, for example, an adenovirus comprising a heterologous nucleic acid sequence means one or more heterologous nucleic acid sequences.

The use of the term “or” in the claims is used to mean “and/or” unless explicitly indicated to refer to alternatives only or the alternatives are mutually exclusive, although the disclosure supports a definition that refers to only alternatives and “and/or.”

The term “antigen” as used herein refers to a substance against which an immune response is generated. The antigen reacts with the products of an immune response stimulated by a specific immunogen, including both antibodies and/or T lymphocyte receptors. Examples include, sugars, lipids, intermediary metabolites, hormones, complex carbohydrates, phospholipids, nucleic acids, and proteins. As used herein, the term antigen includes epitopes of antigens.

The term “immunogen” as used herein refers to a substance that is able to induce a humoral antibody and/or cell-mediated immune response.

The term “immunogenic” refers to the capacity to induce humoral antibody and/or cell-mediated immune responsiveness.

The term “biologically active” in the context of nucleic acids encoding biologically active molecules or products refers to products that exert some physiological effect on the subject to which they are administered. Thus, reporter molecules such as β-galactosidase, luciferase, and green fluorescent protein are not biologically active. Examples of biologically active products include, but are not limited to, cytokines, tumor antigens, viral antigens, bacterial antigens, interfering RNA molecules, etc.

The terms “treating,” and “treatment,” as used herein, refer to both therapeutic and preventative (prophylactic) measures described herein. These terms are inclusive of procedures that prevent, cure, slow disease progression, delay onset of, reduce the severity of, or ameliorate one or more symptoms of a disorder. For example, using the methods of the invention, an effective amount of adenovirus can be administered to a subject as a treatment for a hyperproliferative disorder such as cancer. “Treatment” can also mean prolonging survival as compared to expected survival if not receiving treatment. Those in need of treatment include those already with the condition or disorder as well as those prone to have the condition or disorder or those in which the condition or disorder is to be prevented.

As used herein, the term “tumor” refers to all neoplastic cell growth and proliferation, whether malignant or benign, and all pre-cancerous and cancerous cells and tissues. For example, a particular cancer may be characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue. However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

As used herein, the term “genetically modified” refers to cells or viruses that have been manipulated to contain a non-native (heterologous) polynucleotide (e.g., a transgene) by recombinant methods (e.g., an induced mutation such as IKKβ). For example, cells can be genetically modified by introducing a nucleic acid molecule that encodes a selected polypeptide.

As used herein, the term “transgene” refers to a polynucleotide (e.g., DNA or RNA) that is inserted into a cell or vector and that encodes an amino acid sequence corresponding to a polypeptide. For example, the encoded polypeptide may be capable of exerting a therapeutic or regulatory effect.

As used herein, the terms “protein” or “polypeptide” includes proteins, functional fragments of proteins, and peptides, whether isolated from natural sources, produced by recombinant techniques or chemically synthesized. Typically, the polypeptides typically comprise at least about 6 amino acids, and are preferably sufficiently long to exert a biological or therapeutic effect.

As used herein, the term “vector”, such as an adenoviral vector, means a construct, which is capable of delivering, and preferably expressing, one or more gene(s) or sequence(s) of interest in a host cell. Examples of vectors include, but are not limited to, viral vectors (e.g., adenoviral vectors), naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, the term “expression control sequence” means a nucleic acid sequence that directs transcription of a nucleic acid sequence. An expression control sequence can be a promoter, such as a constitutive or an inducible promoter, or an enhancer. The expression control sequence is operably linked to the nucleic acid sequence to be transcribed. For example, the CMV promoter may be utilized.

As used herein, the term “nucleic acid sequence” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally-occurring nucleotides.

As used herein, the term “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

The terms “replication-deficient” and “replication-defective” mean that the adenoviral vector comprises a genome that lacks at least one replication-essential gene function. A deficiency in a gene, gene function, gene, or genomic region, as used herein, is defined as a deletion of sufficient genetic material of the viral genome to impair or obliterate the function of the gene whose nucleic acid sequence was deleted in whole or in part. Replication-essential gene functions are those gene functions that are required for replication (i.e., propagation) of a replication-deficient adenoviral vector. Replication-essential gene functions are encoded by, for example, the adenoviral early regions (e.g., the E1, E2, and E4 regions), late regions (e.g., the L1-L5 regions), genes involved in viral packaging (e.g., the IVa2 gene), and virus-associated RNAs (e.g., VA-RNA I and/or VA-RNA II). Preferably, the replication-deficient adenoviral vector comprises an adenoviral genome deficient in at least one replication-essential gene function of one or more regions of an adenoviral genome (e.g., two or more regions of an adenoviral genome so as to result in a multiply replication-deficient adenoviral vector). The one or more regions of the adenoviral genome are preferably selected from the group consisting of the E1, E2, and E4 regions. More preferably, the replication-deficient adenoviral vector comprises a deficiency in at least one replication-essential gene function of the E1 region (denoted an E1-deficient adenoviral vector), particularly a deficiency in a replication-essential gene function of each of the adenoviral E1A region and the adenoviral E1B region. In addition to such a deficiency in the E1 region, the recombinant adenovirus also can have a mutation in the major late promoter (MLP), as discussed in International Patent Application WO 00/00628. More preferably, the vector is deficient in at least one replication-essential gene function of the E1 region and at least part of the nonessential E3 region (e.g., an Xba I deletion of the E3 region) (denoted an E1/E3-deficient adenoviral vector).

Optionally, the adenoviral vector can be deficient in replication-essential gene functions of only the early regions of the adenoviral genome, only the late regions of the adenoviral genome, or both the early and late regions of the adenoviral genome. The adenoviral vector also can have essentially the entire adenoviral genome removed, in which case it is preferred that at least either the viral inverted terminal repeats (ITRs) and one or more promoters or the viral ITRs and a packaging signal are left intact (i.e., an adenoviral amplicon). The larger the region of the adenoviral genome that is removed, the larger the piece of exogenous nucleic acid sequence that can be inserted into the genome. For example, given that the adenoviral genome is 36 kb, by leaving the viral ITRs and one or more promoters intact, the exogenous insert capacity of the adenovirus is approximately 35 kb. Alternatively, a multiply deficient adenoviral vector that contains only an ITR and a packaging signal effectively allows insertion of an exogenous nucleic acid sequence of approximately 37-38 kb. Of course, the inclusion of a spacer element in any or all of the deficient adenoviral regions will decrease the capacity of the adenoviral vector for large inserts of heterologous nucleic acid sequences. Suitable replication-deficient adenoviral vectors, including multiply deficient adenoviral vectors, are disclosed in U.S. Pat. Nos. 5,851,806; 5,994,106; and 6,482,616; and International Patent Applications WO 95/34671 and WO 97/21826.

### II. EXAMPLES

Following are examples that illustrate procedures for practicing the invention. These examples should not be construed as limiting. All percentages are by weight and all solvent mixture proportions are by volume unless otherwise noted.

### Example 1

### Generation and Testing of Adenovirus that Expresses a Constitutively-Activated Form of the Human NF-κB Activating Kinase, IKKβ (Ad-IKK)

A replication-deficient adenovirus was generated that expresses a constitutively-activated form of the human NF-κB activating kinase, IKKβ (Ad-IKK). Mutations in two key serine residues (S177 to E, S181 to E) render the IKKβ constitutively-activated. Constitutively activated IKKβ was cloned into shuttle vectors (Zheng Y. et al., J. Exp. Med., 2003, 197:861-874). The expression cassette was then transferred into the replication-deficient (regions E1- and E3-deficient) human type 5 adenovirus vector (Vector BioLabs, Philadelphia, Pa.). A CMV promoter was used for driving expression of IKKβ. The recombinant adenoviral DNA was linearized and transfected into 293 cells for initial viral production. This virus was then amplified in Ad-293 cells and purified using CsCl gradients.

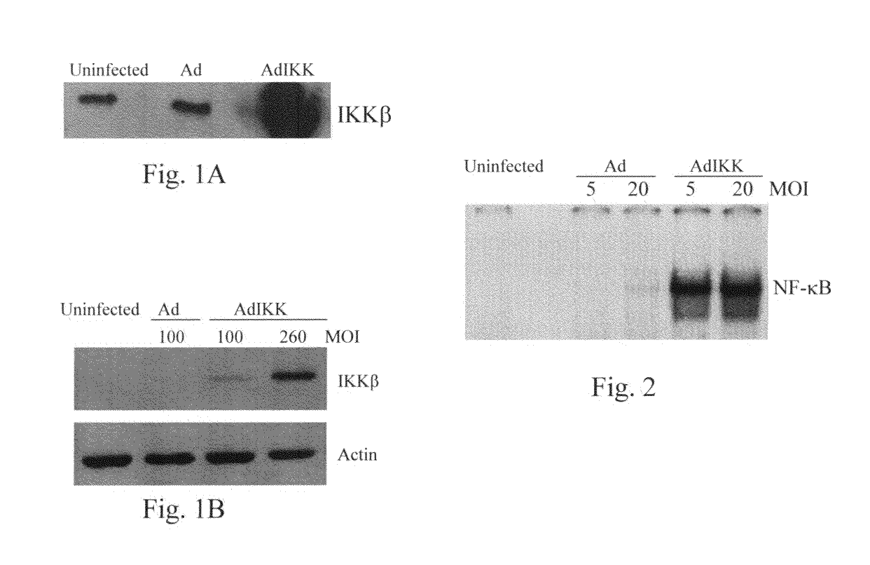

First, whether IKKβ is indeed expressed by Ad-IKK needed to be verified. HEK cells were infected with Ad-IKK and a control GFP expressing adenovirus (Ad). Cell extracts were collected and subjected to western blotting using an IKKβ-specific antibody. As shown in FIG. 1A, a dramatic increase in IKKβ expression was seen following infection with Ad-IKK but not following infection with Ad. Similar results were obtained following infection of mouse dendritic cells (FIG. 1B). Importantly, infection with Ad-IKK induced robust activation of NF-κB in HEK cells while Ad did not induce appreciable NF-κB activation (FIG. 2). Thus, Ad-IKK infection is sufficient to activate the NF-κB transcription factor.

### Example 2

### Effect of Ad-IKK Infection on Expression of Immuno-Stimulatory Molecules in Dendritic Cells

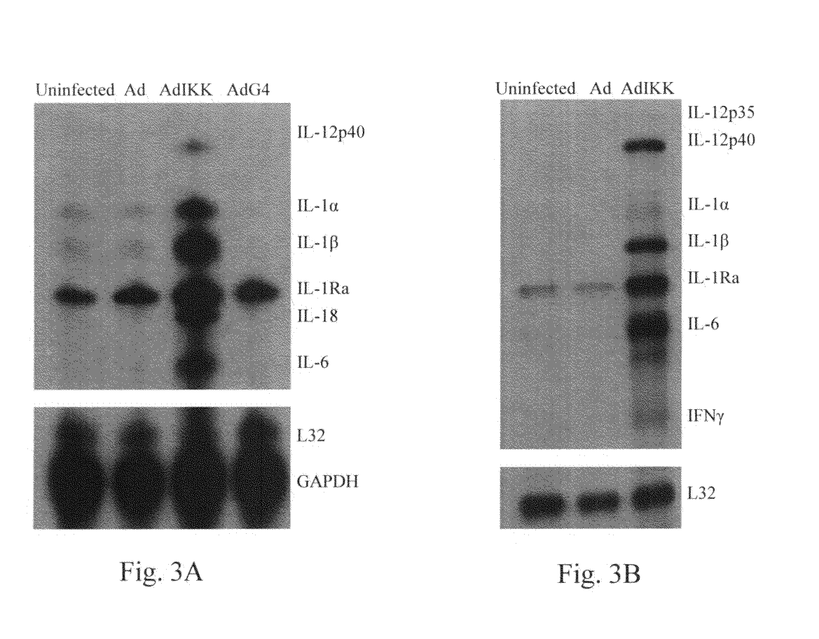

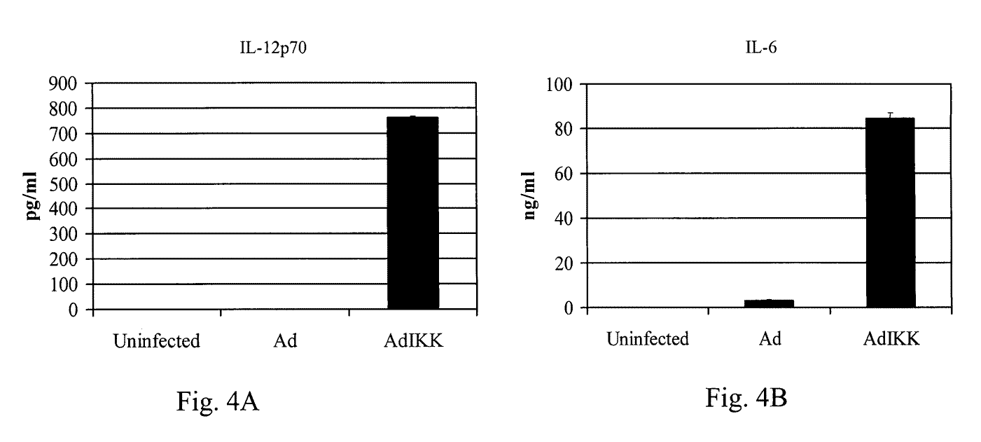

Ad-IKK is able to stimulate immunity. Ad-IKK is able to infect dendritic cells (DCs) and induce expression of genes that are crucial for generating immune responses. DCs are the most important antigen-presenting cell-type and critical for activation of T cells, which regulate both cell-mediated and humoral immune responses. Mouse DCs obtained from the C57BL/6 strain were infected with Ad-IKK or control Ad virus. Importantly, Ad-IKK, but not Ad, strongly stimulated expression of immuno-stimulatory molecules in these DCs, including IL-12, IL-Iα, IL-1β, IL-6 (shown in FIG. 3A). In addition, Ad-IKK infection-induced expression of immuno-stimulatory genes is robust enough to be evident in DCs from different mouse strains, and most important, human DCs. Indeed, similar results were obtained when Ad-IKK was used to infect BALB/c mouse strain DCs (data not shown) and human DCs generated from donor blood (shown in FIG. 3B). Furthermore, protein expression of immuno-stimulatory genes is enhanced following Ad-IKK infection. Protein expression of two key cytokines, IL-6 and IL-12, was strongly enhanced by Ad-IKK but not by Ad (FIGS. 4A and 4B). Remarkably, Ad-IKK enhanced expression of these cytokines more strongly than the microbial agent lipopolysaccharide (LPS), considered one of the strongest inducers of these genes. Thus, Ad-IKK meets important requirements for a vaccine, and can be utilized to induce protective immunity against infectious agents and generate anti-tumor immune responses.

Thus, Ad-IKK is useful to express target antigens against which an immune response is desirable. Based on in vitro studies, Ad-IKK induces a considerably stronger immune response against target antigens than an adenovirus that does not express IKK. The most advantageous aspect of this system is that any target Ag can be expressed in Ad-IKK, including those encoded by infectious agents or tumor cells. Therefore, there is tremendous potential and utility for use of Ad-IKK. Importantly, Ad-IKK efficacy is available even when not expressing target antigens. For example, local administration (e.g., direct injection) of Ad-IKK to tumors creates a more immuno-stimulatory environment and thus can enhance the ability of T cells or other cell-types to kill tumor cells.

### Example 3

### Expression of Constitutively-Activated IKKβ is Sufficient for Induction of Inflammatory Cytokine Genes

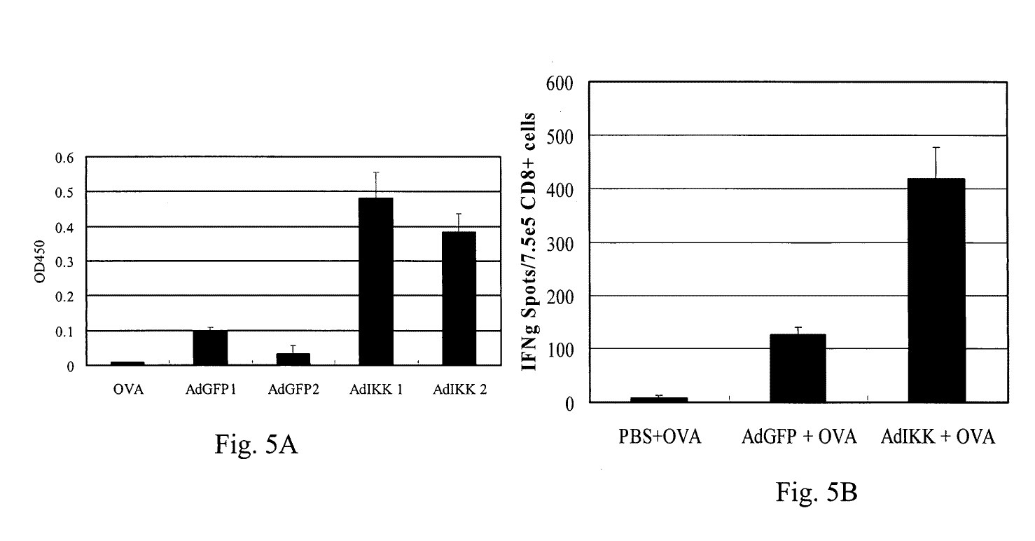

Ad-IKK was constructed to express GFP for identification of infected cells. Compared to control Ad-GFP, Ad-IKK induced robust activation of NF-κB in 293 cells. In addition, Ad-IKK, but not Ad-GFP, was sufficient to induce expression of inflammatory and immune response genes, including IL-12, IL-1α, IL-1β, IL-6, TNFα and CD40 (data not shown). The levels of cytokine mRNAs induced by Ad-IKK were remarkably similar to those induced by LPS. The typical percentage of DC infection at an MOI of 200 was greater than 60% for both Ad-GFP and Ad-IKK (data not shown). Protein expression of immunostimulatory cytokines by ELISA showed expression of IL-6 and IL-12 was strongly enhanced by Ad-IKK but not by Ad-GFP (data not shown). Thus, expression of CA-IKKβ is sufficient for expression of inflammatory cytokines, and therefore, different levels of IKKβ/NF-κB activation by different stimuli can lead to induction of distinct inflammatory gene expression responses. To determine whether Ad-IKK (or Ad-GFP) can act as an adjuvant when co-administered with an antigen, mice were immunized s.c. with Ad-IKK or Ad-GFP together with the model antigen ovalbumin (ova). Both viruses stimulated the anti-ova antibody response compared to ova alone (FIG. 5A). However, Ad-IKK stimulated Ab responses significantly more strongly than Ad-GFP. The effect of these viruses on cell-mediated immunity was then determined by monitoring expansion and functional differentiation of ova-specific CD8 T cells by IFNγ ELISPOT. Similar to Ab responses, both viruses stimulated ova-specific CD8 T cells, with Ad-IKK inducing ˜4-fold greater expansion than Ad-GFP (FIG. 5B).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. An adenoviral vector comprising a recombinant adenovirus that expresses a constitutively activated form of human NF-κB activating kinase (IKK), and expresses a heterologous nucleic acid sequence encoding a biologically active product.

2. The adenoviral vector of claim 1, wherein the IKK is IKKβ, and the adenovirus comprises mutations in serines at amino acid residues 177 and 181 that render the IKKβ constitutively activated.

3. The adenoviral vector of claim 2, wherein the mutations are substitutions comprising S177E and S181E, or S177D and S181D.

4. The adenoviral vector of claim 1, wherein the IKK is IKKα, and the adenovirus comprises mutations in serines at amino acid residues 176 and 180 that render the IKKα constitutively activated.

5. The adenoviral vector of claim 4, wherein the mutations are substitutions comprising S176E and S180E, or S176D and S180D.

6. The adenoviral vector of claim 1, wherein the adenovirus is human adenovirus type 5.

7. The adenovirus of claim 1, wherein the biologically active product is a polypeptide.

8. The adenovirus of claim 1, wherein the biologically active product is an antigen of an infectious agent, a tumor antigen, or an interfering RNA molecule.

9. An immunogenic composition comprising:
(a) (i) a recombinant adenovirus that expresses a constitutively activated form of human NF-κB activating kinase (IKK), and (ii) an antigen, immunogen, and/or adjuvant; or
(b) a recombinant adenovirus that expresses a constitutively activated form of human NF-κB activating kinase (IKK), and expresses a heterologous nucleic acid sequence encoding a biologically active product; or
(c) an immuno-stimulatory cell comprising a recombinant adenovirus that expresses a constitutively activated form of human NF-κB activating kinase (IKK).

10. The immunogenic composition of claim 9, wherein the antigen of (a) (ii) is an immuno-stimulatory microbial product.

11. The immunogenic composition of claim 9, wherein the antigen of (a) (ii) is a protein.

12. The immunogenic composition of claim 9, wherein the biologically active product is an antigen.

13. The immunogenic composition of claim 9, wherein the immuno-stimulatory cell is a dendritic cell.

14. The immunogenic composition of claim 9, wherein the adenovirus of (a) and (c) also expresses a heterologous nucleic acid sequence encoding a biologically active or non-biologically active product.

15. The immunogenic composition of claim 14, wherein the heterologous nucleic acid sequence encodes a biologically active product, and the biologically active product is a protein antigen.

16. A method for inducing an immune response in a mammal, comprising:
(a) administering an effective amount of an adenovirus comprising a mutation that expresses a constitutively activated form of human NF-κB activating kinase (IKK), or
(b) administering an effective amount of immuno-stimulatory cells comprising an adenovirus comprising a mutation that expresses a constitutively activated form of human NF-κB activating kinase (IKK),
wherein an immune response is induced in the mammal.

17. The method of claim 16, wherein the induced immune response comprises a cell-mediated immune response, humoral immune response, or both.

18. The method of claim 16, wherein the adenovirus further comprises a heterologous nucleic acid sequence.

19. The method of claim 18, wherein the nucleic acid sequence encodes a biologically active molecule.

20. The method of claim 19, wherein the nucleic acid sequence encodes an antigen of an infectious agent, a tumor antigen, or an interfering RNA molecule.

21. The method of claim 16, further comprising administering an antigen, immunogen, and/or adjuvant to the mammal before, during, or after administration of the adenovirus or immuno-stimulatory cells.

22. The method of claim 20, further comprising administering an antigen, immunogen, and/or adjuvant to the mammal before, during, or after administration of the adenovirus or immuno-stimulatory cells.

23. The method of claim 21, wherein the antigen is protein antigen.

24. The method of claim 21, wherein the adenovirus, or the immuno-stimulatory cells, and the antigen, immunogen, and/or adjuvant are administered to the mammal simultaneously, within the same composition.

25. The method of claim 16, wherein the mammal is human.

26. The method of claim 16, wherein the immuno-stimulatory cells are human dendritic cells.

27. A method of producing an adenoviral vector, comprising: providing an adenovirus; inducing mutations in the adenovirus such that the adenovirus expresses a constitutively activated form of human NF-κB activating kinase (IKK); and introducing a heterologous nucleic acid sequence into the adenovirus, wherein the heterologous nucleic acid sequence encodes a biologically active product.

28. The method of claim 27, wherein the mutations are substitutions of the serines at positions 177 and 181, or positions 176 and 180.

29. The method of claim 27, wherein the biologically active product is a protein antigen.

30. A method of producing an immunogenic composition, comprising combining: (a) an adenovirus comprising a mutation that expresses a constitutively activated form of human NF-κB activating kinase (IKK), and (b) an antigen, immunogen, and/or adjuvant.

31. The method of claim 30, wherein the adenovirus further comprises a heterologous nucleic acid sequence.

32. A viral particle comprising an adenovirus with expresses a constitutively activated form of human NF-κB activating kinase (IKK), and expresses a heterologous nucleic acid sequence encoding a biologically active molecule.

33. An isolated host cell comprising an adenovirus with a constitutively activated form of human NF-κB activating kinase (IKK), and expresses a heterologous nucleic acid sequence encoding a biologically active molecule.

34. An isolated immuno-stimulatory host cell, comprising an adenovirus with a constitutively activated form of human NF-κB activating kinase (IKK).

35. The isolated immuno-stimulatory host cell of claim 34, wherein the cell is a dendritic cell.
